(19) United States
(12) Patent Application Publication
(10) Pub. No.: US 2015/0366704 A1
(43) Pub. Date: Dec. 24, 2015

(54) Pupil Expansion Device

(71) Applicant: Gregory A. Eippert, Concord, OH (US)

(72) Inventor: Gregory A. Eippert, Concord, OH (US)

(21) Appl. No.: 14/742,850

(22) Filed: Jun. 18, 2015

Related U.S. Application Data
(60) Provisional application No. 62/013,915, filed on Jun. 18, 2014.

(51) Int. Cl. A61F 9/007

(52) U.S. Cl. CPC A61F 9/007; A61F 2250/0059

(57) ABSTRACT

A flexible pupil expansion ring having a U-shaped groove formed along an external diameter of the ring to substantially fully engage and support an iris of a patient's eye, at least one positioning plate extending from a leg of the U-shaped groove, and having an opening within the plate to assist with positioning of the ring with the iris engaged within the groove, and an expansion mechanism for moving the flexible ring from an engagement position to an expansion position enlarging the diameter of the groove and engaged iris.

### CROSS-REFERENCE TO RELATED APPLICATION

This application claims priority to U.S. Provisional Application No. 62/013,915, filed Jun. 18, 2014, the entire contents of which are incorporated herein by reference.

### FIELD OF INVENTION

This device is for use during ocular procedures to assist in dilation of the pupil in situations where pupillary dilation is insufficient for procedures, such as in cataract surgery, or any other situation necessitating additional pupillary dilation for improved visualization of the posterior chamber of the eye.

### BACKGROUND

As our population ages, cataract surgery has become one of the most commonly performed surgeries in the United States, with over 2 million people having the procedure each year. While at an early stage cataracts may be improved with eyeglasses, brighter lighting, anti-glare sunglasses, and magnifying lens or other aids, advanced cataracts, when the loss vision interferes with your daily activities, require surgery. Cataract surgery is generally performed under local or topical anesthesia and involves pre-op, post-op and follow-up care. During surgery, a small corneal or limbal incision is made within a patient's eye. In order to remove the cloudy lens and implant a new artificial lens into the eye, known as an intraocular (IOL) lens, the pupil must be properly dilated to enable a full view of the intraocular tissue, and to avoid damage to the iris and other complications. Unfortunately, pupil dilation can be difficult in this same population due to advancing age and medication related side effects. Providing additional support to the iris beneath the operating incision to help prevent iris prolapse from the wound in situations where such iris muscle laxity is of concern (i.e., Floppy Iris Syndrome), is desired.

### SUMMARY

The device of this application is a flexible circular ring that is able to be expanded to the appropriate size to meet the iris dilation requirements of the surgeon. The device also has the ability to be compressed or folded together to a substantially cylindrical configuration, and placed within an injector or injection tool that is sized to fit through the small corneal/limbal incision made within a patient's eye during the ocular procedure. Once injected or inserted through the incision, the ring is removed from the injection tool and expands into its circular form or position. The device comprises a flexible or compressible polymer ring having a groove, with a generally U-shaped cross-sectional configuration, formed substantially around its entire circumference, with the possible exception of a small portion of the groove located along an expansion mechanism. Once the ring is in the engagement position, with the iris at the pupillary margin engaged to fit securely within the groove, the expansion mechanism of the device is used to allow the ring to expand the pupil uniformly around its 360 degree circumference.

Areas called positioning plates extend from the ring at locations around the ring, and extend further than the grooved border, both anteriorly and posteriorly. These plates assist in positioning the ring and capturing the iris securely within the groove. The device further includes an expansion mechanism that will allow it to be expanded to its appropriate and desired size once introduced and positioned at the iris border. An expansion tool, such as expansion forceps, is used to expand the ring to its proper size to securely engage the iris and expand it to its desired diameter. Markings may be provided on the ring to indicate specific measured expansion diameters. Once the ocular procedure is complete, the ring may be easily disengaged from the iris by reversing the steps described and retracting the ring to its insertion condition and back through the incision due to its ability to compress or fold together as it did during implantation.

The device is a disposable device made of a flexible polymer material, such as medically appropriate silicone, acrylic or other appropriate flexible medical polymer. A unique inserter/injection system is provided that accomplishes the goals of inserting the device in a compact condition and expanding the ring to its proper and desired size.

These and other features of the device will be better understood through the following detailed description and accompanying drawings.

### BRIEF DESCRIPTION OF THE DRAWINGS

FIG. 1 schematically illustrates a top view of the pupil expansion device of the present application continuously engaged with, supporting and expanding an iris of a patient's eye.

FIG. 2 illustrates a perspective view of the pupil expansion device of FIG. 1 shown in collapsed or folded condition and positioned within a cylindrical insertion or injection system device prior to insertion through an incision into a patient's eye, and the pupil expansion device is shown engaged with an expansion tool, a portion of which is also positioned within the insertion or injection device.

FIG. 3 schematically illustrates a cut-away perspective view of a portion of the pupil expansion device having a U-shaped groove on an external diameter of the ring for support and engagement with an iris of a patient's eye, and an internal portion for positioning and housing of the expansion mechanism for enabling the pupil to be expanded uniformly around its 360 degree circumference by expansion and contraction of the ring of the pupil expansion device during positioning on the iris.

FIG. 4 illustrates a perspective view of an embodiment of the pupil expansion device ring including multiple positioning plates extending outwardly further than the groove, both anteriorly and posteriorly, and with one slightly larger footplate with an opening, and positioned at the most proximal portion on the ring, directly under the operating incision to provide additional support of the iris beneath the operating incision to help prevent iris prolapse.

FIG. 5 illustrates a front view of an alternate embodiment of the pupil expansion device with adjustment openings shown near the groove for engagement by the expansion tool for slidably moving a portion of the ring into and out of engagement with another portion of the ring using an expansion mechanism.

FIG. 6 illustrates a side view of the device embodiment taken along the line 6-6 as shown in FIG. 5.

FIG. 7 illustrates a front view of an alternate embodiment of the pupil expansion device.

### DETAILED DESCRIPTION

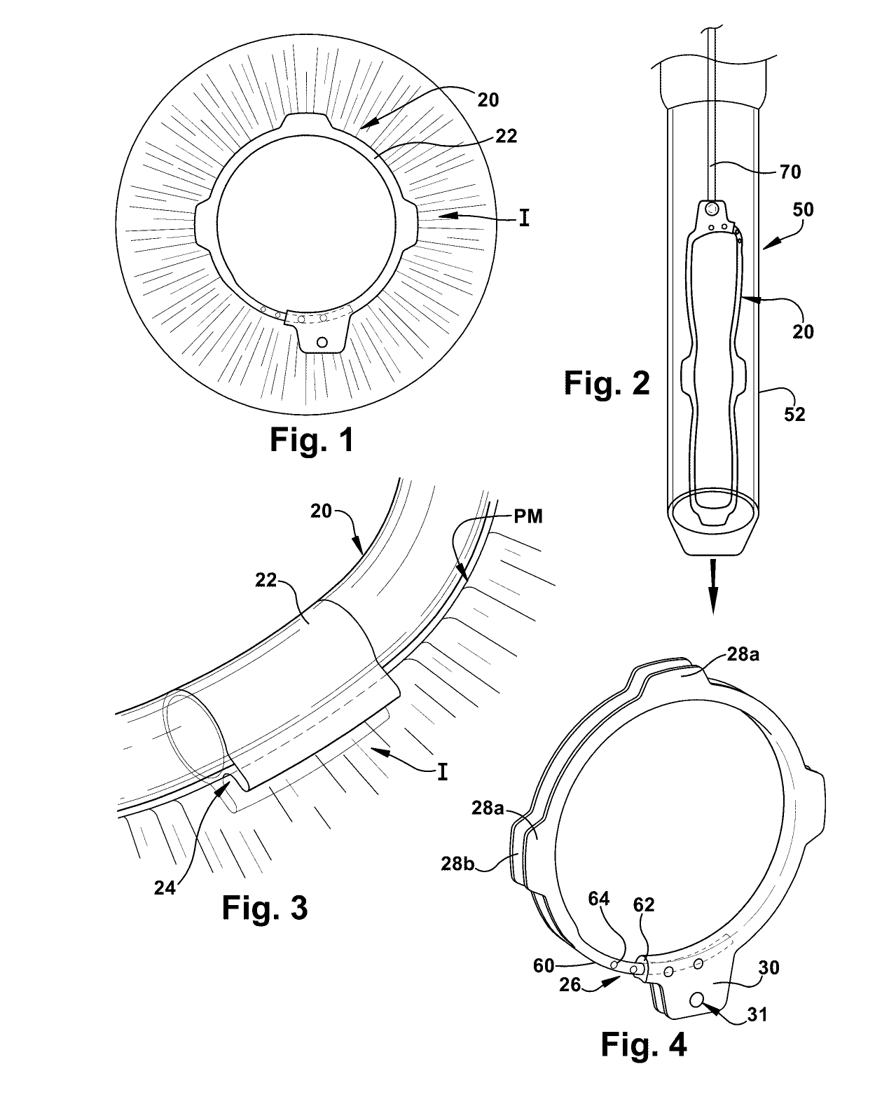

As shown in the attached Figures, the pupil expansion device 20 is a flexible circular ring 22 that is able to be expanded to the appropriate size to meet the iris dilation requirements of the surgeon, as shown expanding the iris I in FIG. 1. As shown in FIG. 2, the device 20 is sufficiently flexible such that it has the ability to be compressed or folded together to a substantially cylindrical configuration, and placed within an injector or injection tool 50 that is sized to fit through the corneal/limbal incision. It is generally understood that such incisions are approximately 2.2-3.2 mm, so that the outside diameter of a cylinder 52 forming the injection tool 50 may range from 2-3 mm in order to fit through the small incision.

Once injected or inserted through the incision and placed inside the eye, the ring is removed from the injection tool 50, as indicated by the arrow, and will expand into its circular form or position. The device 20 includes a ring 22 having a groove 24, with a generally U-shaped smooth cross-sectional configuration, formed substantially around its entire circumference, with the possible exception of a portion of the groove located along an expansion mechanism 26. Once the ring 22 of the device 20 and the iris I at the pupillary margin PM are engaged with the iris I fitting securely within the groove 24, the expansion mechanism 26 is used to allow the pupil to be expanded uniformly around its 360 degree circumference. The external diameter of the ring is approximately 4-5 mm in its non-expanded size, but is able to expand to up to about 9 mm. Thus, the ring 22 allows for expansion of the pupil of between 4-9 mm in diameter. The ring itself is approximately 2 mm thick. The groove for engagement with the iris is preferably approximately 2-5 mm in depth.

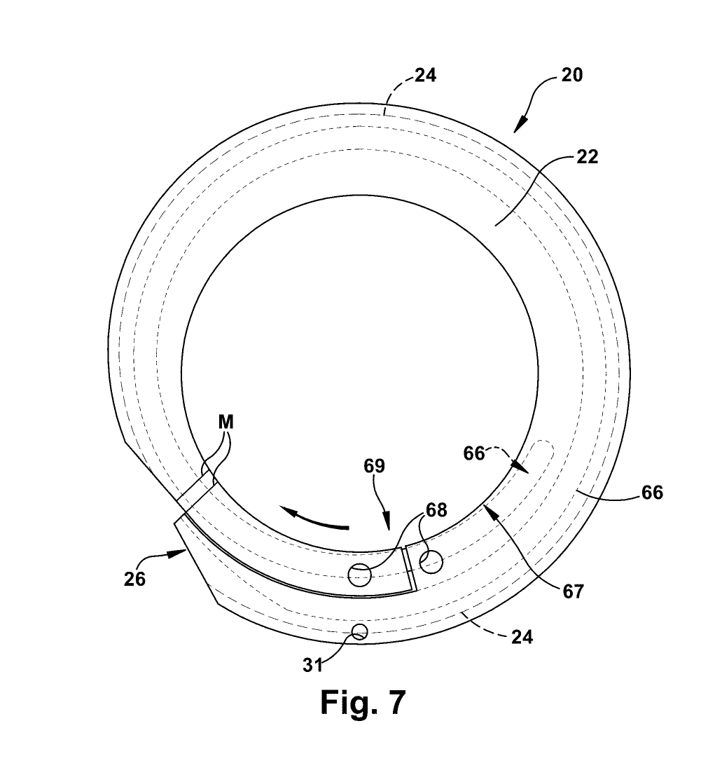

Areas called positioning plates 28 extend in the manner of a boss from the ring at locations around the ring, and extend further outwardly from the groove of the ring, both anteriorly 28a and posteriorly 28b. These plates 28 will serve as areas to assist in positioning the ring 22 and capturing the iris I securely within the groove 24 during the procedure. The ring 22 has a larger footplate or plate 30, having an opening 31, and located at the most proximal portion on the ring, directly under the intended operating incision. This plate 30 will provide additional support for the iris I beneath the operating incision to help prevent iris I prolapse from the wound in situations where iris muscle laxity is of concern (i.e., Floppy Iris Syndrome). While plates 28 at 4 locations are illustrated, it should be understood that no plates, as shown in FIG. 7, 2 plate locations or as many as 8 plate locations are also optional. Additionally, it should be understood that the device 20 may be manufactured in multiple sizes, to address small, medium and large iris sizes.

The device 20 includes an expansion mechanism 26 also near the proximal portion of the ring 22 that allows the ring to be expanded to its appropriate and desired dilation size once introduced and positioned at the iris border PM. As shown in FIG. 4, the expansion mechanism 26 may take the form of a narrower diameter end 60 of the ring 22 telescoping into a larger diameter tube-like end 62. In this embodiment, teeth (not shown) may be formed on the smaller diameter end 60 to engage mating teeth on the internal portion of the larger diameter tube-like end 62, such that sliding engagement of the smaller end 60 into the larger tube-like end 62 engages the teeth to resist disengagement. This engagement retains the ring in the desired open position once it is enlarged to the desired size surrounding, supporting and engaging the iris. Alternatively, the expansion mechanism may include openings 64 formed in the narrow diameter end 60, which may be blocked with a pin or forceps tool to prevent movement of the expansion mechanism 26.

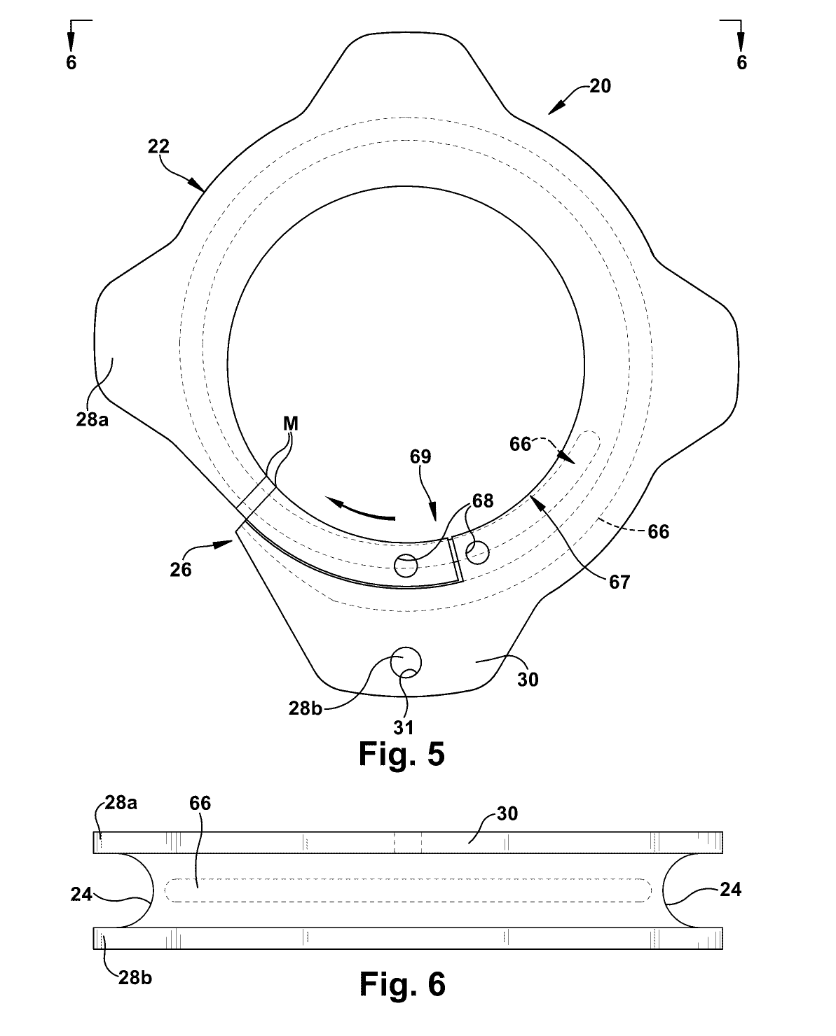

Still further alternative expansion mechanisms 26, as shown in FIGS. 5 and 6, include an additional internal substantially stiff member 66, such as 0.2 mm Nitinol medical grade wire, which is flexible or elastic and slidably engaged along and within an internal passage 67, and in part embedded within a moving end portion 69 of the ring 22, for expanding the ring 22 to the desired expanded dilation diameter in the direction of the arrow shown in FIG. 5. Internal rigid member material alternatives preferably include shape memory retention characteristics to enable folding of the ring 22 to the compressed position within the insertion device 50, and upon removal, to provide return movement to the engagement and expansion positions. Adjustment openings 68 are provided within the ring 22 such that expansion tools 70, for example, expansion forceps or Sinskey hooks, are used to expand the ring to its proper size to securely engage the iris and expand it to its desired diameter. Markings M may be provided on the ring to indicate specific measured diameters. Once the ocular procedure is complete, the ring will be easily disengaged from the iris by reversing the procedures described, such that the ring 22 is contracted to disengage from the iris I, compressed or folded to a cylindrical configuration, and retracted back through the incision, again due to its ability to compress or fold together as it did during implantation.

The device will be a disposable device made of a flexible polymer material, such as medically appropriate silicone, for example, overmolded clear silicone with a 50 A durometer hardness, or an acrylic or other appropriate flexible medical polymer. The inserter/injection system 50 is provided to accomplish the goals of inserting the device 20 in a compact condition, shown as folded together or collapsed on itself in FIG. 2, which upon insertion and removal from the system 50, the ring 22 expands or is expanded and returns to its proper and desired size.

Although the pupil expansion device of the present application has been described in detail sufficient for one of ordinary skill in the art to practice the invention, it should be understood that various changes, substitutions and alterations may be made without departing from the spirit or scope of the device as defined in the attached claims. Moreover, the scope of the present device is not intended to be limited to the specific embodiments described here, which are provided by way of example. As one of ordinary skill in the art will readily appreciate from the disclosure of the present device and its embodiments, other components and means presently existing or later to be developed that perform substantially the same function to achieve substantially the same result as those of the corresponding embodiments described here, may be utilized according to the present application. Accordingly, the appended claims are intended to include within their scope such other components or means.

I claim:

1. A collapsible ring which is initially inserted into a patient's eye through a narrow incision in a substantially collapsed cylindrical insertion condition, and is expanded using an expansion mechanism into a circular support ring having a groove formed along an external diameter of the ring to substantially fully engage with an iris of a patient's eye, and using at least one positioning plate extending from the ring to assist with expansion of the ring with the iris engaged within the groove.

2. A method for dilating a pupil of a patient's eye comprising the steps of:
inserting a collapsible ring from a collapsed condition within an insertion tube, through an incision, and out of the tube into the interior of an eye,
engaging an iris secured within a U-shaped groove formed along an external diameter of the ring in an engagement position; and
expanding the ring to expand the iris diameter using an expansion mechanism housed within the ring in an expansion position.

3. A flexible polymer pupil expansion ring having a U-shaped groove formed along an external diameter of the ring to substantially fully engage and support an iris of a patient's eye, at least one positioning plate extending from a leg of the U-shaped groove, and having an opening within the plate to assist with positioning of the ring with the iris engaged within the groove, and an expansion mechanism for moving the flexible ring from an engagement position to an expansion position enlarging the diameter of the groove.

4. The ring of claim 1 or 3 wherein the expansion mechanism includes an internal adjustable support member for moving the ring between an engagement position having a diameter for engagement of the groove with an iris, and an expansion position having an enlarged diameter for expansion of the pupil with the groove supported and engaged with the iris.

5. The ring of claim 1 or 3 wherein the expansion mechanism includes an embedded and slidably adjustable wire for moving the ring between an insertion condition, to an engagement position and an expansion position.
